(19) United States
(12) Patent Application Publication
(10) Pub. No.: US 2008/0102514 A1
(43) Pub. Date: May 1, 2008

(54) REACTOR FOR THE CULTURE, BIOOXIDATION OF SOLUTIONS AND/OR LARGE-SCALE PROPAGATION OF ISOLATED MICROORGANISMS AND/OR NATIVE MICROORGANISMS THAT ARE USEFUL IN ORE LEACHING

(75) Inventors: Roberto Coallao Olivares, Providencia (CL); Leandro Mauricio Padilla Iglesias, Providencia (CL); Pedro Antonio Morales Cerda, La Reina (CL); Ricardo Badilla Ohlbaum, La Reina (CL)

Correspondence Address: MERCHANT & GOULD PC, P.O. BOX 2903, MINNEAPOLIS, MN, 55402-0903, US

(73) Assignee: BIOSIGMA S.A., Colina (CL)

(21) Appl. No.: 11/862,026

(22) Filed: Sep. 26, 2007

(30) Foreign Application Priority Data
Oct. 27, 2006 (CL) 2912-2006

(51) Int. Cl. C12N 1/00; C12M 1/00

(52) U.S. Cl. 435/262; 435/303.3

(57) ABSTRACT

The invention publishes a reactor and method for the culture, biooxidation of cations in solution and/or large-scale propagation of jointly isolated microorganisms, with or without native microorganisms that are useful in sulfide metal ore bioleaching. The invention particularly publishes a reactor for the large-scale culture and/or propagation of an association of Acidithiobacillus thiooxidans Licanantay DSM 17318 isolated microorganisms jointly with Acidithiobacillus ferrooxidans Wenelen DSM 16786 with or without the presence of other microorganisms.

### BACKGROUND OF THE INVENTION

The invention publishes a reactor for the large-scale culture and/or propagation of micro-organisms isolated jointly with or without native microorganisms, that are useful in ore metal sulphide leaching. The reactor is also used for bio-oxidizing, through the action of extremophylic microorganisms that oxidize cations such as ferrous ion, arsenious ions, cuprous ions or others contained in effluents, liquid industrial residues or other solutions of industrial interest in overall treatment, confinement and metal recovery processes. The invention specially publishes a reactor for large scale culture and/or propagation of an association of Acidithiobacillus thiooxidans Licanantay DSM 17318 isolated micro-organisms together with Acidithiobacillus ferrooxidans Wenelen DSM 16786, with or without other microorganisms.

### SUMMARY OF THE INVENTION

Over 90% the world's mine copper is currently obtained from copper sulfide ore processing. The most important copper sulfide species present in ores are chalcopyrite, bornite chalcosite, covellite, tenantite and enargite, of which chalcopyrite is the species found in most relative abundance, and therefore the one of greatest economic interest.

Copper sulfide ore processing nowadays is sustained by technologies based on physical and chemical processes associated with mineral crushing, grinding and flotation, followed by fusion-conversion of concentrates and electrolytic refining of metal. In practice, over 80% of copper is produced through the described route—known as conventional—which is limited to high and medium grade ores, according to the specific characteristics of deposits and of ore processing plants. For this reason, there are vast and extensive resources of relatively low grade ores, which with conventional technologies are sub-economic, and remain unexplored for lack of an effective technology with which to work them.

On the other hand, ores in which copper is present in the form of oxide species (easily soluble in acid) are processed by means of acid leaching processes, followed by solvent extraction processes and electro-winning of the metal, in what is known as copper winning through hydrometallurgy. This route is very attractive due to its lower operation and investment costs when compared to conventional technologies, as well as to its lower environmental impact. Nevertheless, applications of this technology are limited to oxide ores, or to copper sulfide mixed ores in which metal is present in the form of secondary sulfides (chalcosite and covellite) that are acid soluble in the presence of an energetic oxidizing agent catalyzed by microorganisms (Uhrie, J L, Wilton, L E, Rood, E A, Parker, D B, Griffin, J B and Lamana, J R, 2003, “The metallurgical development of the Morenci MFL Project”, Copper 2003 Int Conference Proceedings, Santiago, Chile, Vol VI, 29-39).

It has been established for a long time that sulfide ore solubilization or leaching is helped by the presence of iron and sulfur oxidizing bacteria, which is known as bioleaching (for example see the recent review by Rawlings D E; Biomineralization of metal-containing ores and concentrates, TRENDS in Biotechnology, Vol. 21 No. 1, p38-42, 2003). When working these metals with commercial-scale bioleaching in heaps or dumps using mesophylic microorganisms at temperatures ranging from 25-45° C., satisfactory recoveries and extraction speeds of 80% recovery in 270 days of operation, for bio-leaching secondary sulfides such as covellite (CuS) and chalcosite (Cu₂S), are obtained. Within this temperature range, the bacteria present most widely described are of the Acidithiobacillus and Leptospirillum genres, of which the most common species are A. ferrooxidans, A thiooxidans, and L. ferrooxidans (Espejo R T and Romero, J., 1997, “Bacterial community in copper sulfide ores inoculated and leached with solutions from a commercial-scales copper leaching plan”, Applied & Environmental Microbiology, Vol 63, 4, 183-187).

According to the above, ways of favoring growth conditions for microorganisms that participate in bioleaching are mentioned in several processes. For example, WO2004027100, sets forth a method in which exopolymer-free microorganisms are produced and later injected into a bioleaching heap where they are provided with the nutrients and/or conditions they need to generate these exopolymers. Document WO0071763 expounds the introduction of an acid solution containing bacteria into the leaching heap. Another document, US 20040091984, mentions the incorporation of bacterial cultures obtained from leaching ponds to favor bioleaching. WO03068999 sets forth that the use of liquid inoculums poses problems associated with an unequal distribution, and using aerosols is proposed as a solution.

Even though the documents mentioned above mention the incorporation of microorganisms into leaching heaps, no references or descriptions of reactors for culturing microorganisms are found in which they are presented as necessary.

On the other hand, in documents such as U.S. Pat. No. 6,110,253, in which thermophylic microorganisms are added to the heap on isolated occasions, the need for practical industrial methods for culturing large quantities of bacteria is not taken into account.

In a different approach such as the one in U.S. Pat. No. 5,763,259, enrichment of the ore's own bacterial flora which is worked by dehydrating particles of this ore on which the bacteria grow and even by lowering the activity of the remaining water, is carried out. With this approach, microorganism culture is carried out in essentially solid phase, and the inoculum proposed is also solid. This document doesn't present many details of the reactor in which this enrichment process is carried out either.

Similarly, document RU2188243 expounds treating part of the ore in a reactor, particularly with sulfooxidizing bacteria. The ore is subsequently removed from the reactor and mixed with the other part of the ore, and this mixture is then heaped up. In this case there isn't a description of the reactor either, and furthermore it can also be stated that it is high-cost equipment mainly because it must treat over 5% of the total ore processed.

Finally, document CL 42.561 presents a bio-electrochemical reactor that makes it possible to obtain high microorganism densities for bioleaching sulfide ores by means of electrochemical regeneration of the ferrous ion, applying electric energy from a continuous power source regulated according to the oxide-reducing potential. Air is injected into this reactor as well, to provide both carbon dioxide and oxygen which are necessary for the bacteria. According to this document, in order to obtain high cell densities it is necessary to use external electric energy to regenerate the ferrous ion. Nevertheless, the bio-electrochemical reactor presents severe technical limitations, especially at membrane-level, for up-scaling to industrial level, and for this reason it is not used in commercial systems.

Just as it can be observed, based upon the quoted documents, in technics there is major concern regarding the increase in the number of active microorganisms in ores in order to enhance bioleaching, and particularly regarding the increase of a certain type of microorganism, a type that depends on the bioleaching practiced. This can be explained with two reasons:

Firstly, the microorganisms present in the ore, or their kinetics, may not be the most appropriate for the bioleaching conditions, which explains the inoculation of specific microorganisms.

Secondly, starting sulfide copper bacterial bioleaching requires that the bacteria come into contact with the surface of the ore to be bio-leached, and then multiply so as to colonize the surface of the available solid. Once colonization has occurred, bioleaching kinetics become faster (Lizama, H. M., Fairweather, M. J., Dai, Z., Allegretto, T. D. 2003a. “How does bioleaching start?”. Hydrometallurgy. 69: 109-116).

To this effect, a latency or “lag” phase during which ore dissolution kinetics are slow has been observed in bioleaching pilot operations, a fact that has been associated with the stage in which the ore surface is colonized by the microorganisms (Lizama, H M; Harlamovs, J R; Belanger, S; Brienne, S H. 2003b. “The Teck Cominco HydroZinc process”. Hydrometallurgy 2003: 5th International Symposium Honoring Professor Ian M. Ritchie; Vancouver, BC; Canada; 24-27 Aug. 2003. pp. 1503-1516. 2003).

Therefore, if there were a reactor available for the large-scale culture and/or propagation of an adequate source of microorganisms, for example, for the continuous inoculation of heaps and/or dumps, it would be possible to shorten the phase in which the ore is colonized by bacteria, and/or a high concentration of bioleaching bacteria on the ore surface could be obtained.

The effects or having this source of microorganisms available would then be, on one hand, shortening of the lag phase which in turn means reducing total bioleaching time, and on the other hand, this microorganism source would make an increase of microorganisms in the ore possible, resulting in faster bioleaching of the ore.

Now, from the point of view of underlying biology, it is known that bioleaching microorganism growth is sensitive to parameters such as temperature, pH, composition of the solution, and aeration, among others, over which there is little control in a heap or dump, and which also vary according to their location within the system, and may therefore be far from the optimum conditions that can be obtained in a reactor in which there is more control over these parameters.

Furthermore, culturing strains of interest, in heaps and dumps, such as a particular mesophyll or thermophile strain, competes with the growth of native microorganisms. Therefore the common practice in “in situ” culture industrial operations, that is to say right in heaps, dumps or tailings dams or in other similar operations, has the disadvantage of making control over microorganism culture very difficult if a microorganism concentration lower than that of the reactors used for similar processes is obtained, and if it does not specifically favor microorganism species that present a higher capacity to bioleach the interesting ores. (Ojumu, T V, Petersen, J, Searby, G E, Hansford, G S, 2005, “A review of rate equations proposed for microbial ferrous-iron oxidation with a view to application to heap bioleaching” Proceedings of the 16th International Biohydrometallurgy Symposium, Cape Town, South Africa, Vol VI, 85-93; Brierley, C L., 2001, “Bacterial Succession In Bioheap Leaching” Hydrometallurgy 59, 249-255).

These two reasons: control of growth conditions and competition with other microorganisms, make culturing microorganisms that are useful in bioleaching in reactors with controlled conditions interesting, thus providing optimum culture conditions, and decreasing competition with other less interesting microorganisms.

Just as it can be observed, industrial practice in bioleaching operations in piles and dumps doesn't consider controlled production of microorganisms that are useful in this bioleaching, at a scale appropriate for the problem, and could be advantageously used to diminish the ore colonization phase, or increase the concentration of microorganisms in this ore. Therefore, as far as we know, we can state that the need for a culture system is maintained, suach as for example, a reactor with controlled conditions, that will allow the large-scale continuous culture and/or propagation of microorganisms useful in bioleaching of ores.

For a better understanding of the processes linked to the continuous, controlled generation of inoculum, the following concepts are outlined below:

Continuous operation: It consists in the continuous generation of a bacterial inoculum with which heaps and/or dumps are irrigated. This flow of inoculum is generated in a reactor into which a similar flow of culture medium enters, and whose operating conditions are controlled.

Batch operation: Prior to the continuous generation of inoculum, it is necessary to reach an appropriate concentration of bacteria in the reactor, which is achieved by means of the batch operation of the reactor during a period in which bacteria growth, up to this concentration level, is achieved.

Culture Medium Aqueous solution containing the salts that contribute elements that make up the bacteria biomass (nutrients), as well as the source of energy required for their growth.

Energy source: Compound used by bacteria as a source of energy for their growth and maintenance. In the case of ferro-oxidizing bacteria this source may be ferrous, and in the case of thiooxidizing bacteria they are reduced sulfur compounds. It is possible to generate a mixture of ferro-oxidizing and thiooxidizing bacteria in these very reactors by using a source of energy appropriate for both types of bacteria, such as pyrite, or else a mixture of energy sources.

In order to have a large quantity of isolated microorganisms, that are useful in sulfide metal ore leaching, using bioreactors and controlled conditions, a reactor that allows the large-scale propagation of biomass, which can be used in sulfide metal species bioleaching, has been developed. This reactor is a particular bioreactor, that allows the continuous production of different types of microorganisms, such as for example, Acidithiobacillus ferrooxidans and Acidithiobacillus thiooxidans jointly, with or without native microorganisms.

### BRIEF DESCRIPTION OF THE DRAWINGS

In order to make understanding this invention easier, the following figures were used:

FIG. 1: Shows the elevation of the reactor for continuous generation of inoculum of the present invention. In this FIG. 1, number (1) represents the cylindrical body of the reactor, and number (2) represents the reactor base, number (3) represents the reactor cover, number (4) represents the coil, number (5) represents the entrance of fluid into the coil (4), number (6) represents the exit of fluid from the coil (4), number (7) represents the entrance of the mixture of air and CO₂, numbers (8, 9) represent the pipes that feed the mixture of air and CO₂, number (10) represents the aerators, number (11) represents the exit from the reactor to the secondary stirring system, number (12) represents the entrance to the reactor from the secondary stirring system, number (13) represents the entrance of basic pH solution, number (14) represents the entrance of acid pH solution, number (15) represents the entrance of culture medium, number (16) represents the entrance of energy source, number (17) represents the entrance of inoculum, number (18) represents the air vent, number (19) represents inoculums exit, number (20) represents exit for sample taking and number (21) represents the reactor drainage.

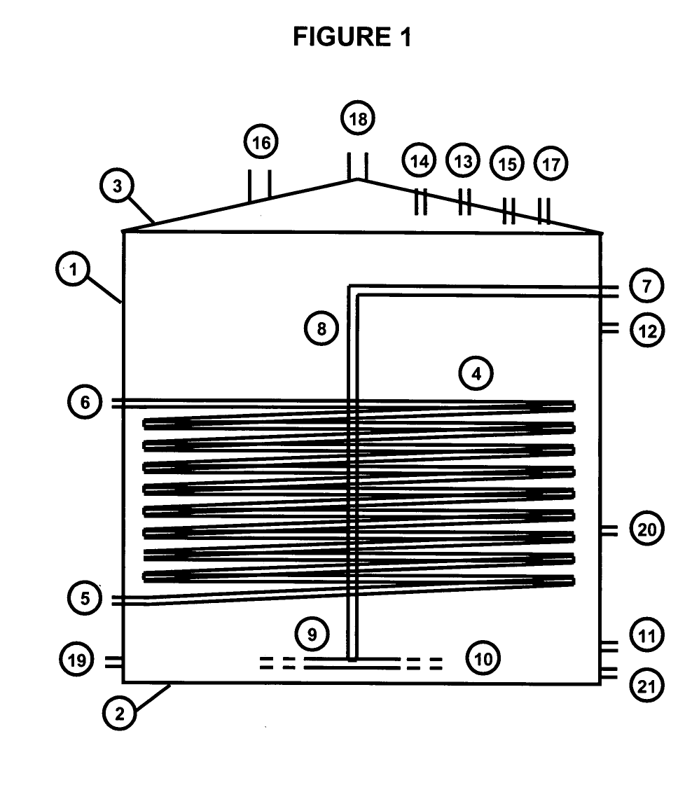

FIG. 2: Shows the elevation of the reactor for the continuous generation of inoculum, of the present invention. In this FIG. 2, number (1) represents the reactor's cylindrical cover, and number (2) represents the base of the reactor, number (3) represents the cover of the reactor, number (22) represents the manhole, number (23) represents a combined sensor for temperature and dissolved oxygen, number (24) represents a potential Eh sensor, number (25) represents a pH sensor and number (26) represents a reactor contents level sensor.

FIG. 3: Plan view of upper part or cover of the reactor. In this figure, number (3) represents the cover of the reactor, number (13) represents the basic pH solution entrance, number (14) represents the acid pH solution entrance, number (15) represents the culture medium entrance, number (16) represents the energy source entrance, number (17) represents the inoculum entrance, and number (18) represents the reactor air vent.

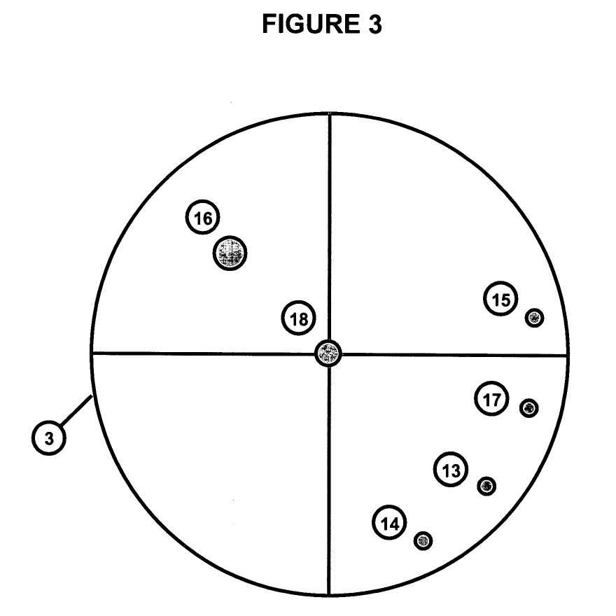

FIG. 4: Shows the plan view of the reactor interior, below the coil (4) (which is not seen in this figure) and above the base (2), showing the layout of the air and CO₂ mixture distribution system at the base of the reactor. In this figure, number (2) represents the base of the reactor, number (8) represents a 4 to 8-inch diameter stainless steel feed pipe, number (9) represents a 4 to 6 inch nominal diameter stainless steel feed pipe, and number (10) represents aerators, for example MaxAir model 00865 type.

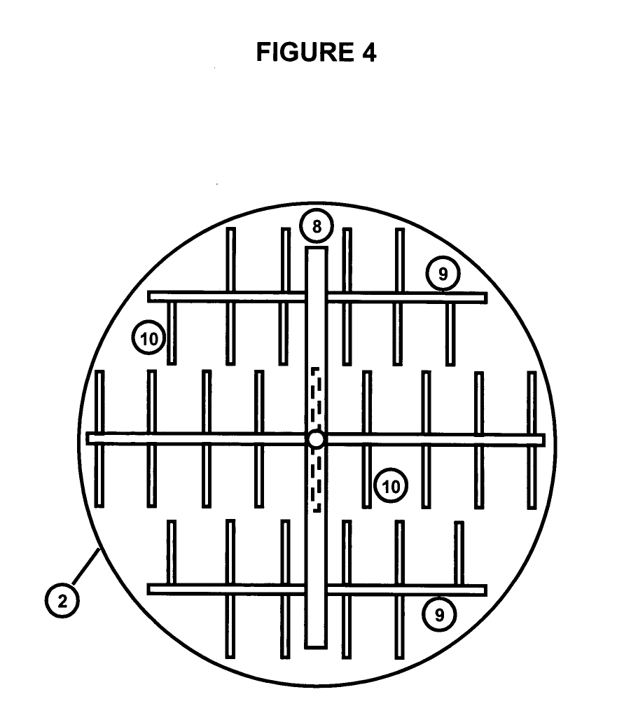

### DETAILED DESCRIPTION OF THE PREFERRED EMBODIMENTS

The reactor for the continuous production of inoculum, according to the present invention, consists in a bubbling column type reactor, with a cylindrical body, composed of a cylindrical body (1) and a base (2). The reactor is closed with a cover (3) which allows the reactor to be covered, and also allows materials that may fall on the reactor to slide off it, preventing these materials from remaining on it. This makes it possible to prevent foreign substances from failing into the reactor, it allows the reactor to be installed in the open air, and it also makes lowering the construction cost of the cover possible, by preventing its having to support the weight of materials that could accumulate on it.

The construction materials of the reactor, as well as the construction materials of all the elements that operate or are used in the reactor interior, as for example, coils, baffles, supports, etc., are fit for acid environments, which means that they can, for example, stay in contact with solutions with pHs as low as 1.0. Materials that among others are considered appropriate, are fiber glass reinforced with polyester using phenolic or alquidic resin, and stainless steel.

Regarding reactor geometry, a reactor body height/reactor diameter relationship of around 2.0 is considered appropriate, for example from 1.5 to 2.5; in special cases, the relationship can reach 3.0.

On the other hand, regarding the geometry of the reactor cover or roof (3), conical geometry is considered to be adequate, and for this reason, the diameter of the base is identical to that of the reactor casing, or slightly larger, and the relationship of the reactor casing height to the height of the conical shape that forms the cover, is from 9 to 11.

The operation level of the reactor, in other words the level up to which its liquid contents are allowed to reach, doesn't coincide with the height of the reactor, and is lower. On one hand, this provides leeway for the volume of air and CO₂ which at that moment are in the gas phase and circulate within the reactor, and on the other hand provides a security margin to prevent the reactor from overflowing. This way, the reactor's total height is 10% to 25% more than the height corresponding to the liquids it contains during its normal operation.

Due to different conditions, such as for example, environmental conditions, that will depend on the place and situation in which the reactor is installed, and the optimum reaction temperature which ranges from 25 to 35° C., it is necessary that the reactor also have an internal temperature modifying system. This system is materialized as a series of conduits, such as for example, tubes that make it possible to make a fluid such as a gas or liquid at a temperature higher, lower or equal to the desired reactor operating temperature, circulate. This conduit system can be found inside the reactor, for example, taking the shape of a coil that runs through a part of the inside of the reactor, or all the inside of the reactor, whether at its center or on the periphery, or can be installed on the outside of the reactor, like a jacket that partially covers it or includes its base and cover. In a preferred presentation, this system of conduits takes the shape of a coil (4) installed in the reactor's interior. As it has been previously mentioned, a fluid flows through this conduit system, providing or taking away heat, if its temperature is respectively higher or lower than the temperature inside the reactor, and this fluid can be a gas such as water vapor, some chlorofluorocarbon or fluorocarbon, a liquid such as hot or cold water, or any other fluid considered to be appropriate. This fluid enters the conduit system by an entrance (5) and comes out of it by an exit (6), both of which in a preferred presentation are found in the casing (1) of the cylindrical reactor.

The reactor is also provided with a system that permits the entry of a mixture of air and CO₂, a mixture that provides:
a) Oxygen required by the bacteria as final acceptor of electrons, during the consumption of the energy source needed by the bacteria to grow and remain.
b) CO₂ required by the bacteria as a source of carbon.
c) Stirring required to homogenize the reactor contents.
d) In cases in which the energy source of the bacteria consists in solid particles, the air flow allows these particles to stay in suspension.

The purpose of using a CO₂-enriched mixture is to achieve an increased bacterial growth rate, so the reactor is provided with a system that allows the air entering the reactor to be enriched, increasing the concentration of CO₂. This is achieved by incorporating CO₂ into the column of air impelled towards the reactor. The point where CO₂ is incorporated can be found before the impelling system or after the impelling system. This depends on conditions such as the CO₂ pressure provided, the system that controls the proportion of air to CO₂, and/or other conditions of the particular setup.

The mixture of air and CO₂ is put into contact with the reactor contents and impelled through an entrance (7) from the reactor exterior to its interior, and then through a piping system (8,9), and the mixture of air and CO₂ is finally impelled by a system of aerators (10) arranged inside the reactor, near its base, that provide both bubbling enough for transferring the oxygen required, and the bubbling necessary for mixing the contents of the reactor. The aerators (10) used can be fine or large bubble aerators, and are built of a material that can stay in acid environments, for example, with a pH as low as 1.5. This material can be polyester reinforced glass, using phenolic resin for the aerator body with a teflon or viton membrane, or a stainless steel tube with perforations appropriate for the type of bubble desired. The distribution system for the mixture of air and CO₂ is also provided with a conduit system (8, 9) that permits the air and CO₂ mixture to arrive from outside the reactor to its interior, passing through the entrance (7), and makes it possible to keep the same pressure in all the aerators (10), so that this mixture is uniformly distributed in all the base of the reactor, providing air and CO₂ and stirring all the contents of this reactor. Just as it has been previously mentioned, the construction material of these conduits must be capable of remaining in contact with solutions with a pH as low as 1.5, and for this purpose some of the materials considered appropriate are fiber glass and stainless steel.

The mixture of air and CO₂ is driven towards the reactor by means of a positive displacement blower, such as a lobular or screw blower, driven by a motor, for example an electric motor or an internal combustion motor fed with gasoline, petroleum, alcohol or gas, and provided with a system that enables its rotation speed to vary, for instance, if an electric motor is used, a frequency variator, or in the case of an internal combustion motor, a mechanism that will allow the feeding of fuel to the motor to be varied, which, as it has been explained, makes it possible to vary the motor rotation speed, and thus operate with different flows of the air and CO₂ mixture. As previously mentioned, there is not only one type of appropriate positive displacement blower, but the choice will depend particularly on the air and CO₂ flow discharge pressure, which in turn is linked to the height of the reactor water column. Just as an example, and in increasing order of discharge pressure, lobular blowers and screw blowers may be mentioned.

Varying the flow of the air and CO₂ mixture makes it possible to adjust power consumption on the part of the motor that drives the blower, in order to comply with a predetermined level of oxygen and/or CO₂ inside the reactor. This control is achieved by establishing a control loop between two or more dissolved oxygen and/or CO₂ sensors arranged, for example, inside or on the bottom of the reactor, and by the frequency variator of the motor that drives the blower or the fuel feeding mechanism, depending on which motor is used.

On the other hand, the flow variation of the air and CO₂ mixture also makes it possible to act on the agitation level of the reactor contents, which will depend, among other conditions, on cell density, quantity of material in suspension, density of suspended material, etc. So, if greater stirring is required, the rotation speed of the motor that drives the blower can be increased to increase the flow, so that agitation inside the reactor increases in turn. Analogically, if the agitation level is excessive, it is possible to decrease the flow of air acting on the speed control mechanism of the motor that drives the blower. This control mechanism can be established by means of a control loop between, for example, gauging of density, viscosity, cell count and the previously mentioned motor speed control mechanism.

As people experimented in bubble column type reactor technique know, the variables previously mentioned, on one hand the concentration of dissolved oxygen and/or dissolved CO₂, and on the other hand the reactor agitation level, are controlled in order to lower power costs, but they are not possible to control independently. In practice, it is found that the rotation speed of the motor that drives the blower is the one that simultaneously satisfies both variable types, the concentration of dissolved O₂ and/or CO₂, and the degree of stirring. This speed is normally the highest among the ones that satisfy the conditions separately.

Added to the above, and in view of the possibility of solid decantation occurring due to different problems in the aeration system, as for example, that for a specific level of solids in the reactor the air flow required for the suspension of the materials amply surpasses the recommended levels of oxygen and/or CO₂ or simply means exceedingly high power consumption, the reactor is also provided with a secondary system to stir its contents. This is achieved by recirculating the reactor contents that are at the bottom of it towards the surface, with which these solids are kept in suspension. In order to achieve this objective, the reactor is provided with a pipe for recirculating located on its exterior, a pipe that is in fluid communication with a pump, such as for example a screw pipe or diaphragm pump. As it was previously explained, the pump entrance is communicated with the bottom of the reactor or with a point near its base where the exit (11) from the reactor to the secondary stirring system is located, whereas the exit from the pump is communicated with the upper part of this pump, at a point at or above its operation level, where the entrance (12) from the secondary stirring system is. As for the pump employed, two requirements are mandatory, first that it be capable of impelling materials with high solid contents, and second, that it not produce shear in excess of what the microorganisms cultured inside the reactor can tolerate. As an example, two types of pumps that satisfy these requirements are screw pumps and diaphragm pumps, and among the latter, those that are directly driven or the ones driven by employing an air current.

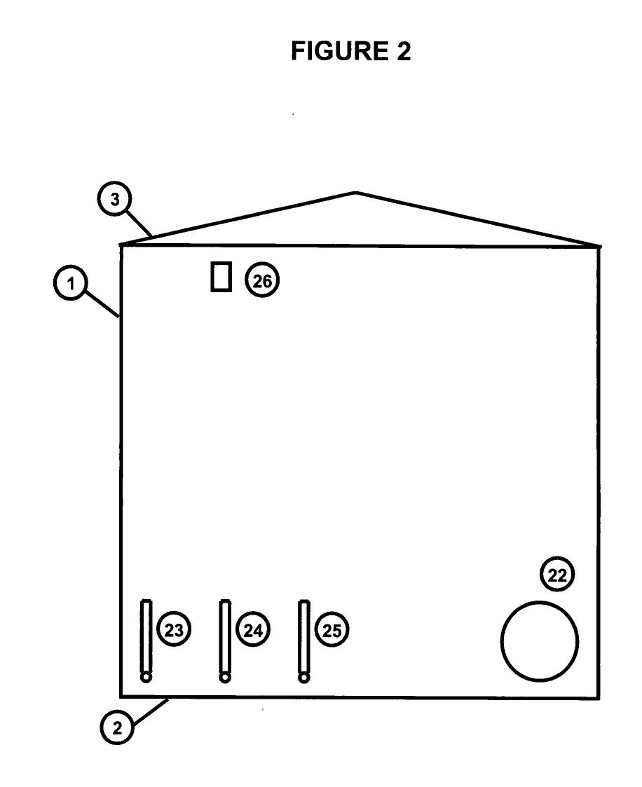

The reactor is also provided with entrances for fluids that make it possible for the pH to be controlled. There are two of these entrances, an entrance (13) for fluid with a basic pH, such as a sodium hydroxide solution, and an entrance (14) for a fluid with an acid pH, such as sulfuric acid. The fluids are impelled by pumps, which can be, for example, piston or diaphragm proportioner pumps, which are controlled by means of a closed loops with one or more pH electrodes arranged inside the reactor with an exit towards the exterior (24 and 25, FIG. 2).

The reactor is also provided with a culture medium adding system. This system consists in an entrance (15) for culture medium which is fed to the reactor by gravity or impelled by means of a pump. In order to control and/or know the quantity of culture medium impelled towards the reactor, a fluid gauging system can be used, such as for example a full tube flow sensor, or a controlled proportioner pump can be used.

The reactor is also provided with a system for adding energy source, a source that may be in a solid or liquid state when added. This system consists in an (16) entrance to the reactor, through which this energy source is fed. As in the previous case, in order to know and/or control the quantity of energy source added to the reactor, a gauging system can be used such as for example a flow sensor if dealing with an energy source in a liquid state, or for example a scale installed on a feeding conveyor belt if dealing with an energy source in a solid state.

The reactor is also provided with an entrance (17) for inoculum, which allows it to be fed continuously or in portions from another production facility. As in previous cases, the inoculum can be impelled by means of an appropriate pump, as for example a diaphragm pump powered by electricity or by air pressure, or a screw pump. Furthermore, in order to know the quantity of inoculum driven towards the reactor, as in previous cases, it is possible to drive the inoculum by means of a pump that also complies with the proportioning characteristics, or by installing a flow gauging system, such as a full tube flow sensor, or something similar.

In order to allow volumes impelled towards the reactor, such as inoculum volume, culture medium volume, pH controlling reagent volume, and energy source volume to force out the air within the reactor, the reactor is also provided with an air vent (18) located in the reactor cover (3).

The reactor is also provided with an exit (19) for continuous inoculum produced in the reactor, which consists of a solution with a cellular density typically ranging from 1×10⁷ to 1×10⁹ bacteria per ml according to the operating conditions.

The reactor is also provided with an exit (20) for sample taking, and with a drainage (21) located near or at its base, for cases in which it is necessary to empty it completely, like for example when doing general upkeep work. The reactor is also provided with a manhole (22).

As people experimented in the technique will understand, a reactor such as the one described can be operated both in a continuous manner and in lots or batch mode. In a preferred presentation, the reactor was operated in batch mode during the first stage, with the purpose of obtaining a specific concentration, for example 1×10⁹ cells/ml, and subsequently, the mode was changed to continuous operation, to provide a stream of inoculum continuously, with a concentration similar to the one pointed out.

As previously mentioned, for the reactor to operate automatically, it is also provided with several sensors that make it possible to know and/or control the different process variables. Some of the sensors that can be mentioned are for instance, dissolved oxygen sensors (23), temperature sensors (23), Eh potential sensors (24), pH potential sensors (25), liquid level sensors (26), air flow sensors and continuous inoculum flow sensors.

The setup and use of the sensors may be redundant, that is to say, two or more sensors of the same type may be provided, for example, two pH sensors placed at the same point of the reactor, or at different points. There may be several reasons for setting up redundant sensors, for instance, as a security measure, so that if one of the sensors fails, the reactor can continue to operate while the faulty sensor is being replaced, using the working sensor to control the reactor, or it may be an additional control tool, for instance, to evaluate the agitation level indirectly. Furthermore, sensors may be individual or combined. For example, it is common for dissolved oxygen sensors or pH sensors to also incorporate a temperature sensor, which would avoid having to set up an individual temperature sensor, or if it were installed, this measure would be redundant.

The reactor is also provided with an online data acquisition system, which makes it possible to record operation variables, such as for example, temperature, pH, dissolved oxygen, liquid level, and air and inoculum flows. Recording these variables, along with a specific control logic, make it possible in turn to control the reactor by means of at least the following control loops:
a) PH control loop. According to the pH (25) value, the addition of basic pH solution or acid pH solution is triggered by acting on the respective pumps.
b) Temperature control loop. According to information coming from the temperature sensor (23), it acts on and varies the flow of the heating or cooling fluid flow passing through the coil (4).
c) Dissolved oxygen control loop. According to the value of dissolved oxygen in the reactor, it acts on the frequency variator of the motor that drives the blower, which makes the flow of the air and CO₂ mixture entering the reactor vary.

The present invention also publishes the large-scale culture and/or propagation of jointly isolated microorganisms with or without native microorganisms that are useful in the bioleaching of metallic ores by means of the reactor previously mentioned, without limiting the invention and considering that the method may vary according to the needs of the bacteria that are propagated, a method that can be defined as follows:
a) partially filling the reactor with culture medium;
b) setting the pH control system in motion so as to keep the pH at levels between 1.5 and 2.5;
c) set the temperature control system in motion, so as to keep the temperature at approximately 30° C.;
d) setting in motion the system that supplies the air and CO₂ mixture at levels ranging between 0.5% and 3% CO₂ in the volume;
e) incorporating energy source into the reactor;
f) adding a volume of inoculum of iron-oxidizing and sulfur-oxidizing bacteria alone or combined with native microorganisms;
g) operating the reactor in batch mode until the total volume contained in the reactor reaches a microorganism concentration typically higher than 1×10⁹ cells/ml;
h) changing the operating mode to continuous mode;
i) incorporating culture medium and energy source continuously;
j) removing inoculum from the reactor continuously at a rate similar to that of the incorporation of culture medium;
k) adjusting the incorporation rate of culture medium, inoculum and energy source, so that the microorganism concentration at the inoculum exit is kept at counts typically higher than 1×10⁸ cells/ml.

The reactor of the present invention may be used to propagate or culture any microorganism. Microorganisms preferably cultivated are Wenelen DSM 16786, Licanantay DSM 17318 alone or together with native microorganisms. Depending on the pH needed, it will be adjusted with a solution of NaOH or a solution of H₂SO₄.

The inoculum stream is of 300 to 500 liters per hour. The concentration of bacteria varies typically within the range of 1×10⁸ to 1×10⁹ bacteria per ml. Sensors for PH, dissolved oxygen, potential Eh, liquid level, inoculum flow and others, are on line with a control system so that the variables that may affect the bacteria, are controlled. For instance, temperature is kept at 25 to 30° C., with hot or cold water passing through the coil (4) depending on the case.

1. Reactor for the large-scale continuous culture and/or propagation of isolated and/or native microorganisms useful in bioleaching of ores comprising:
a) it is a closed reactor with a cylindrical shape, with a cylindrical body which has a base, and a height to diameter ratio is 1.5 to 2.5; in special cases, the ratio can reach 3.0;
b) it has a cover with a conical shape, with a diameter at the base the same or slightly larger than the diameter of the reactor, and the ratio of the cylindrical casing height to the height of the conical shape formed by the lid is from 9 to 11;
c) it is provided with a principal system and a secondary system to agitate the contents of this reactor;
d) it has a coil installed in its interior, which allows both heating and cooling of reactor contents by circulating a heating or cooling fluid through the coil, a fluid that enters the coil through the entrance and goes out of the coil through the exit;
e) it has an air and CO₂ mixture distribution system in fluid communication with a piping system;
f) it has aerators installed near the base, and in fluid communication with the air and CO₂ distribution system;
g) it has an exit from the reactor to a secondary stirring system, and an entrance to the reactor from this system;
h) it has an entrance for basic pH solution, an entrance for acid pH solution, an entrance for culture medium, an entrance for energy source, and an entrance for inoculum, and an air vent;
i) it has an exit for inoculum, an exit for sample taking, and an exit for drainage;
j) in the casing that forms the reactor, are the entrance for the fluid that circulates through the coil, the exit for the fluid that circulates through the coil, the entrance for the air and CO₂ mixture, the exit from the reactor to the secondary stirring system, the entrance to the reactor from the secondary stirring system, the exit for inoculum, the exit for sample taking, the exit for drainage, and the manhole;
k) on the cover of the reactor, are the entrance for the basic pH solution, the entrance for the acid pH solution, the entrance for the culture medium, the entrance for the energy source, the entrance for inoculum, and the air vent;
l) in the lower third of the casing that forms the reactor, are the entrance for the fluid that circulates through the coil, the exit from the reactor to the secondary stirring system, the exit for inoculum, the exit for sample taking, the exit for drainage, and the manhole;
m) in the upper third of the casing that forms the reactor, are the exit for the fluid that circulates through the coil, the entrance for the air and CO₂ mixture, the entrance to the reactor from the secondary stirring system;
n) the coil is located around the middle third of the cylindrical volume that forms the reactor;
o) the conduit system for the air and CO₂ mixture runs through the whole cylindrical volume that forms the reactor, from the upper third, to the lower third where the aerators are;
p) it is provided with, pH, dissolved oxygen, and Eh potential sensoring elements, and liquid level sensors;
q) it is provided with elements for determining the flow and/or mass incorporated into the reactor such as acid or basic pH solution, culture medium, inoculum and energy source;
r) it is provided with a dissolved oxygen, pH, Eh potential and reactor volume control system;
s) the pH, dissolved oxygen, and Eh potential sensoring elements are located in the lower third of the cylindrical volume that forms the reactor; and
t) it is provided with a system for controlling the entrance of the air and CO₂ mixture.

2. The reactor, according to claim 1, wherein the total volume is from 10 to 25% larger than the liquid volume used.

3. The reactor, according to claim 1, wherein the construction material of the casing, of the base and of the cover is fiber glass reinforced with polyester using alquidic and phenolic resin.

4. The reactor, according to claim 1, wherein the main stirring system, is the air mixture distribution system.

5. The reactor, according to claim 1, wherein because the secondary stirring system is a system that considers a positive displacement pump capable of impelling fluids with high solid contents without producing more shear than what the bacteria being cultured can tolerate, that is fed from the reactor exit to the secondary stirring system, located at a point at or near the reactor base, and that discharges at a point at or above the reactor operation level, described as the entrance to the reactor from the secondary stirring system.

6. The reactor, according to claim 1, wherein the diameter of the cylindrical shape that forms the reactor cover is identical to the diameter of the cylinder that forms the reactor body.

7. The reactor, according to claim 1, wherein the ratio of the height of the conical shape that forms the reactor cover to the casing of the cylinder that forms the reactor, is approximately 10.

8. The reactor, according to claim 1, wherein the ratio of the total reactor volume to the reactor's useful volume ranges from 1.3 to 1.6.

9. Method for large-scale culturing and/or propagation of jointly isolated microorganisms with or without native microorganisms that are useful in metallic ore bioleaching by means of the reactor of claim 1, wherein it includes;
a) partially filling the reactor with culture medium;
b) starting the pH control system so that the pH is kept at 1.5 to 2.5 with appropriate basic or acid solutions;
c) starting the temperature control system, so that the temperature is kept at around 30° C.;
d) starting the system that supplies the air and CO₂ mixture at levels ranging from 0.5% to 3% CO₂ in the volume;
e) incorporating energy source to the reactor;
f) adding a volume of inoculum of iron-oxidizing and sulfur-oxidizing microorganisms alone or combined with native microorganisms;
g) operating the reactor in batch mode until all the volume contained in the reactor reaches a concentration of microorganisms in excess of 1×10⁸ cells/ml;
h) changing the operating mode to a continuous mode;
i) incorporating culture medium and energy source continually;
j) removing inoculum from the reactor continuously at a rate similar to the culture medium incorporation rate,
k) adjusting the culture medium, inoculum, and energy source incorporation rate so that the microorganism concentration at the inoculum exit is kept at counts above 1×10⁸ cells/ml;
i) controlling the agitation level by varying the flow of the air and CO₂ mixture;
m) carrying out secondary stirring by recirculating the reactor contents, from the bottom of the reactor to the surface of these contents.

10. Method for large-scale culture and/or propagation of microorganisms according to claim 9, wherein the microorganisms cultured are Wenelen DSM 16786, Licanantay DSM 17318 alone or together with native microorganisms.

11. Method for large-scale culture and/or propagation of microorganisms according to claim 9, wherein the solution used to adjust basic pH, is an NaOH solution.

12. Method for large-scale culture and/or propagation of microorganisms according to claim 9, wherein the solution used to adjust acid pH is an H₂SO₄ solution.

13. Method for large-scale culture and/or propagation of microorganisms according to claim 9, wherein the inoculum stream is 300 to 500 liters per hour.

14. Method for large-scale culture and/or propagation of microorganisms according to claim 9, wherein the bacteria concentration is 1×10⁸ to 1×10⁹ bacteria per ml.

15. Method for large-scale culture and/or propagation of microorganisms according to claim 9, wherein the sensors for pH, dissolved oxygen, Eh potential, liquid level, inoculum flow, and others, are on line with a control system.

16. Method for large-scale culture and/or propagation of microorganisms according to claim 9, wherein a temperature between 25 and 30° C., is maintained.
